(19) United States
(12) Patent Application Publication
(10) Pub. No.: US 2024/0335374 A1
(43) Pub. Date: Oct. 10, 2024

(54) COMPOSITIONS FOR ANTIHYPERTENSIVE DRUGS

(71) Applicants: Ninad Deshpanday, Cary, NC (US); David M. Oakley, Todd, NC (US); John R. Cardinal, Wilmington, NC (US); William C. Stagner, Apex, NC (US); Jayaram Reddy Thimmapuram, York, PA (US)

(72) Inventors: Ninad Deshpanday, Cary, NC (US); David M. Oakley, Todd, NC (US); John R. Cardinal, Wilmington, NC (US); William C. Stagner, Apex, NC (US); Jayaram Reddy Thimmapuram, York, PA (US)

(21) Appl. No.: 18/381,775

(22) Filed: Oct. 19, 2023

Related U.S. Application Data
(60) Provisional application No. 63/457,833, filed on Apr. 7, 2023.

(51) Int. Cl. A61K 9/00; A61K 31/401; A61K 47/38

(52) U.S. Cl. CPC A61K 9/0043; A61K 31/401; A61K 47/38

(57) ABSTRACT

The present disclosure provides for nasal compositions for antihypertensive drugs and methods for preparing the compositions. The compositions of the disclosure are particularly suitable for pediatric and geriatric populations, and patients with dysphagia, for treatment of conditions and diseases associated with hypertension, essential hypertension, hypertensive crisis, hypertensive emergency, hypertensive urgency, and other cardiovascular disease by administration, via nasal mucosa.

### CROSS REFERENCE TO RELATED APPLICATIONS

This application claims priority to U.S. Provisional Application No. 63/457,833, filed Apr. 7, 2023, the disclosure of which is incorporated by reference in its entirety into the present application.

### TECHNICAL FIELD

The disclosed subject matter provides for nasal compositions comprising antihypertensive drugs. The compositions of the disclosure are particularly suitable for pediatric and geriatric populations, and patients with dysphagia, for treatment of conditions and diseases associated with hypertension, essential hypertension, hypertensive crisis, hypertensive emergency, hypertensive urgency, and other cardiovascular disease by administration via nasal mucosa.

### BACKGROUND

Hypertension (essential) is a major risk factor in the development of cardiovascular diseases. There are number of oral medicines useful for treatment of high blood pressure but obstacles for the oral delivery of antihypertensive drugs include low oral bioavailability due to first pass hepatic metabolism, low drug solubility, low GI permeability/absorption, and/or drug degradation in the GI tract.

Large numbers of patients with dysphagia have difficulty in swallowing their medications which leads to nonadherence to treatment and treatment failure. It is well known that pediatric and geriatric patient populations suffer dysphagia that results in nonadherence to therapy. Pediatric patients often refuse to take tablets because they are difficult to swallow and oral liquids often have an offensive taste the leads to spiting the liquid out.

Clinically significant dysphagia or difficulty with swallowing is prevalent in the older population (30-40%). Not only is aging a factor for dysphagia, but dysphagia is also associated with medical conditions like cancer, stroke, gastric reflux disease (GERD), chronic obstructive pulmonary disease (COPD), Parkinson's disease, multiple sclerosis, dementia, motor neuron disease, and as side effects of commonly prescribed drugs such as antiarrhythmics, antihistamines, decongestants, diuretics, selective serotonin reuptake inhibitors and others.

Patients suffering from hypertensive crisis, hypertensive emergency, or hypertensive urgency need immediate medical attention and systemic antihypertension drug treatment. Hypertensive emergency is caused by an acute, marked elevation in blood pressure that is associated with signs of target-organ damage. Pulmonary edema, cardiac ischemia, neurologic deficits, acute renal failure, aortic dissection, and eclampsia often result in a hypertensive emergency. In order to get immediate drug effect, the drug is injected directly into the patient's vein. Unfortunately, it is often difficult to find a vein because during hypertensive emergency the veins are constricted and narrow. Blood vessels may rupture due to the high blood pressure causing the tissue to swell, making it difficult to find a vein. Blood flow to peripheral veins like hands, arms and legs may be reduced or disrupted making it difficult to find a site to inject an antihypertensive rescue drug.

As patients with hypertensive crisis, emergency or urgency and acute angina attack have markedly reduced functional ability and extreme restlessness, rapid onset of pharmacological action is of prime importance so that patients can return to normal state and resume their functional activities. Currently, this can effectively be achieved only by parenteral administration of antihypertensive drugs, requiring treatment by a healthcare professional or in a hospital setting, which may not be easily and quickly available.

There is an unmet need for non-parenteral, reliable and convenient to administer dosage forms for antihypertensive drugs.

### SUMMARY

In certain embodiments, the disclosure provides a nasal composition comprising an antihypertensive drug.

In certain embodiments, the composition provides faster systemic drug absorption compared to an oral solution containing the same drug.

In certain embodiments, the composition provides a post-dose lag time to the systemic drug absorption that is at least 30 minutes less than the post-dose lag time to the systemic drug absorption from a marketed oral solution containing the same drug.

In certain embodiments, the post-dose systemic drug absorption provided by the composition at about 5 minutes is at least equal to the post-dose systemic drug absorption from a marketed oral solution containing the same drug at about 1.5 hours to about 2 hours.

In certain embodiments, the composition when dose normalized to a marketed oral solution containing the same drug provides at least 2-fold increase in drug plasma concentration at about 1.5 hours post-dose compared to the marketed oral solution. In certain embodiments, the composition when dose normalized to a marketed oral solution containing the same drug provides about 9-fold increase in drug plasma concentration compared to the marketed oral solution.

In certain embodiments, the composition is a solution, suspension or an emulsion comprising the antihypertensive drug and a buffer. In certain embodiments, the composition exhibits a pH of from about a 4 to 7.5. In certain embodiments, the composition exhibits a pH of from about 6 to about 7. In certain embodiments, the composition exhibits a pH of from about 6.1 to 6.7. In certain embodiments, the composition exhibits a pH of about 6.4.

In certain embodiments, the composition is a nasal solution composition comprising a angiotensin converting enzyme inhibitor.

In certain embodiments, the disclosure provides a nasal composition comprising lisinopril, salts or hydrates thereof and a buffer. In certain embodiments, the composition is an aqueous nasal solution comprising from about 4 mg/100 μL to about 8 mg/100 μL lisinopril dihydrate and a buffer.

In certain embodiments, the composition is an aqueous nasal solution administered as nasal spray or nasal drops.

In certain embodiments, the solution is administered nasally once-a-day in one or both nostrils as single spray or drop/nostril, two sprays or drops/nostril, three sprays or drops/nostril, or four sprays or drops/nostril.

In certain embodiments, the buffer is selected from the group consisting of sodium dihydrogen phosphate; disodium hydrogen phosphate; sodium citrate and citric acid; sodium acetate, acetic acid; maleic acid and salts; carbonic acid, ammonia, carbonate, sodium carbonate, decarbonate sodium, trisodium citrate, lactic acid, maleic acid, tartaric acid, sodium tartrate and salts, glycine, tris (hydroxymethyl) aminomethane, and mixtures thereof.

In certain embodiments, the disclosure provides an aqueous nasal solution comprising an antihypertensive drug and a buffer.

In certain embodiments, the nasal solution further comprises a mucoadhesive thickening agent selected from the group consisting of carboxymethylcellulose, sodium carboxymethylcellulose, methyl cellulose, hydroxypropyl methylcellulose, hydroxypropyl cellulose, hydroxyethyl cellulose, microcrystalline cellulose, and mixtures thereof. In certain embodiments, the mucoadhesive thickening agent is present in an amount of up to about 5% wt/vol.

In certain embodiments, the nasal solution is free of any mucoadhesive thickening agent.

In certain embodiments, the solution further comprises a preservative selected from the group consisting of benzalkonium chloride (BAC), potassium sorbate, methyl paraben, propyl paraben, benzyl alcohol, benzoic acid, phenoxyethanol, chlorobutanol, m-cresol, phenol, thiomersal, and mixtures thereof. In certain embodiments, the preservative is present in an amount of up to about 3% wt/vol.

In certain embodiments, the nasal solution is a preservative free solution.

In certain embodiments, the solution further comprises a tonicity agent selected from the group consisting of sodium chloride, dextrose, glycerin, potassium chloride, mannitol, calcium chloride, glucose, glycine, magnesium chloride, potassium chloride, sorbitol, sucrose, sodium sulfate, and mixtures thereof. In certain embodiments, the tonicity agent is present in an amount of up to about 7% wt/vol of the nasal solution.

In certain embodiments, the nasal solution is free of any tonicity agent.

In certain embodiments, the disclosure provides a nasal powder composition comprising an antihypertensive drug.

In certain embodiments, the nasal powder composition is delivered via powder exhalation delivery system comprising a capsule or a device with a reservoir containing the antihypertensive drug.

In certain embodiments, the nasal composition is a nasal suspension comprising an antihypertensive drug. In certain embodiments, the nasal suspension is administered as nasal spray, nasal drops, or pressurized metered aerosol.

In certain embodiments, the disclosure provides a nasal suspension composition comprising an antihypertensive drug, a buffer, a suspending agent, and a surfactant.

In certain embodiments, the suspending agent is selected from the group consisting of carboxymethylcellulose, sodium carboxymethylcellulose, methyl cellulose, hydroxypropyl methylcellulose, hydroxypropyl cellulose, hydroxyethyl cellulose, microcrystalline cellulose, mixture of microcrystalline cellulose and carboxymethylcellulose sodium, poloxamer, polyvinyl alcohol, polyvinyl pyrrolidone, polyvinyl acetate, polydextrose, pectin, chitosan, acacia, polyethylene glycol, sodium alginate, bentonite, carbomer, carrageenan and, guar gum, alginic acid, carbomer, hectorite, povidone, tragacanth, xanthan gum and mixtures thereof. In certain embodiments, the suspending agent is present in an amount of up to about 5% wt/vol.

In certain embodiments, the surfactant is selected from the group consisting of polysorbate polysorbate 20 (polyoxyethylene (20) sorbitan monooleate), polysorbate 80/Tween (polyoxyethylene (80) sorbitan monooleate), dipalmitoylphosphatidylcholine (DPCC), lecithin, oleic acid, sorbitan esters, ammonium lauryl sulfate, trolamine lauryl sulfate, polysorbate 60, other nonionic or ionic surfactants, and mixtures thereof. In certain embodiments, surfactant is present in an amount of up to about 10% wt/vol of the nasal suspension.

In certain embodiments, the nasal composition is an oil-in-water nasal emulsion comprising an antihypertensive drug.

In certain embodiments, the nasal emulsion composition is administered as a nasal spray or nasal drop.

In certain embodiments, the disclosure provides a nasal emulsion composition comprising an antihypertensive drug, a buffer, and an emulsifying agent.

In certain embodiments, the emulsion is an oil-in-water emulsion.

In certain embodiments, the emulsifying agent is selected from the group consisting of polysorbate polysorbate 20 (polyoxyethylene (20) sorbitan monooleate), polysorbate 80/Tween (polyoxyethylene (80) sorbitan monooleate), dipalmitoylphosphatidylcholine (DPCC), lecithin, oleic acid, sorbitan esters, ammonium lauryl sulfate, trolamine lauryl sulfate, polysorbate 60, other nonionic or ionic surfactants, and mixtures thereof. In certain embodiments, the emulsifying agent is present in an amount of up to about 10% wt/vol of the nasal emulsion.

In certain embodiments, the disclosure provides a method for treating hypertension, the method comprising administering a nasal composition comprising an antihypertensive drug.

In certain embodiments, the disclosure provides a method for treating hypertension in pediatric, adult, and/or geriatric patient, the method comprising nasally administering to the patient a nasal composition comprising an antihypertensive drug.

In certain embodiments, the disclosure provides a method for treating hypertension in a patient with dysphagia, the method comprising nasally administering to the patient a nasal composition comprising an antihypertensive drug. In certain embodiments, the dysphagia is associated with conditions selected from the group consisting cancer, stroke, Parkinson's disease, multiple sclerosis, gastroesophageal reflux disease (GERD), chronic obstructive pulmonary disease (COPD), dementia, motor neuron disease, and side effects of commonly prescribed drugs selected from the group consisting of antiarrhythmics, antihistamines, decongestants, diuretics, and serotonin reuptake inhibitors.

In certain embodiments, the disclosure provides a method of treating hypertension associated with hypertensive crisis, hypertensive emergency or hypertensive urgency in a patient, the method comprising nasally administering to the patient a nasal composition comprising an antihypertensive drug.

In certain embodiments, the disclosure provides a method of treating myocardial infarction, heart failure or cardiovascular disease (CVD) in a patient, the method comprising nasally administering to the patient in need thereof nasal composition comprising an antihypertensive drug.

In certain embodiments, the antihypertensive is drug selected from the group comprising angiotensin converting enzyme inhibitor (ACEi), angiotensin receptor blocker (ARB), diuretic, β-blocker, calcium channel blocker, central α-agonist, peripheral α-antagonist, direct vasodilator, neprilysin inhibitor, and mixtures thereof.

In certain embodiments, the antihypertensive drug is an angiotensin converting enzyme inhibitor selected from the group consisting of benazepril, captopril, enalapril, fosinopril, lisinopril, moexipril, perindopril, quinapril, ramipril, trandolapril, alacepril, cilazapril, delapril, imidapril, spirapril, temocapril, zofenopril, and pharmaceutically acceptable salts and forms thereof.

### BRIEF DESCRIPTION OF DRAWINGS

FIG. 1 compares mean lisinopril plasma concentration-time profile after administration of nasal solution spray composition of the disclosure comprising 12 mg equivalent of lisinopril, one spray (6 mg equivalent lisinopril) in each nostril, and literature values of lisinopril oral solution (10 mg lisinopril).

### DETAILED DESCRIPTION

There is growing interest in developing non-parenteral, reliable and convenient dosage forms of antihypertensive drugs, using administrative routes where the drug is rapidly absorbed into systemic circulation. Oral dosage forms and oral route are the most preferred route of administration for various antihypertensive drugs, but have limitations like first-pass hepatic metabolism, long lag times to systemic drug absorption, and reduced compliance, particularly with patients with dysphagia, psychiatric patients, bedridden patients and uncooperative patients.

A antihypertensive drug absorbed via nasal mucosa can provide required systemic bioavailability with low doses, rapid absorption and possible reduction in side effects.

Nasal mucosa has been considered as potential administration route to achieve faster and higher systemic drug absorption due to its high drug permeability compared to absorption of oral dosage forms via GI tract due to several factors, including lack of pancreatic and gastric enzyme activity, neutral pH of nasal mucus, and less dilution by GI contents. The advantage of nasal drug delivery systems includes fast onset of action due to rapid systemic absorption, and avoidance of first pass metabolism, thereby improving bioavailability and reducing side effects compared to conventional oral dosage forms.

Nasal compositions are herein identified to overcome these significant unmet drug delivery issues faced by a large portion of the patient population. Nasal antihypertensive drug delivery to hypertensive pediatric and geriatric patients, hypertensive dysphagic patients, and patients suffering life-threatening hypertensive crisis, hypertensive emergency or hypertensive urgency is critical to effective treatment and outcome.

The presently disclosed subject matter provides a novel approach for treating hypertension, hypertensive crisis, hypertensive emergency, hypertensive urgency, and other cardiovascular diseases including myocardial infarction and heart failure, while providing and maintaining therapeutic plasma concentration of antihypertensive drug comparable to oral antihypertensive dosage forms. In particular, the disclosure provides nasal compositions comprising antihypertensive drugs suitable for treating hypertension in pediatric, geriatric, and adult patients, patients with dysphagia, patients with hypertensive crisis, hypertensive emergency, hypertensive urgency, and other cardiovascular diseases including myocardial infarction and heart failure.

The compositions of the disclosure provide rapid onset of action, improved bioavailability, and reduced side effects by providing faster systemic absorption of antihypertensive drugs with doses similar to the oral tablet or oral liquid dosage forms. These nasal compositions may also be used as replacement for conventional oral dosage forms, e.g., oral tablets or liquids, for convenience and ease of administration in hypertensive subjects.

### Definitions

The terminology used in the present disclosure is for the purpose of describing particular embodiments only and is not intended to be limiting. Certain terms are discussed below, or elsewhere in the specification, to provide additional guidance in describing the compositions and methods of the disclosure and how to make and use them. Unless otherwise defined, all terms, including technical and scientific terms used in the description, have the same meaning as commonly understood by one of ordinary skill in the art to which this disclosure belongs.

As used herein, the use of the word “a” or “an” when used in conjunction with the term “comprising” in the claims and/or the specification can mean “one,” but it is also consistent with the meaning of “one or more,” “at least one,” and “one or more than one.” Still further, the terms “having,” “including,” “containing,” and “comprising” are interchangeable, and one of skill in the art is cognizant that these terms are open-ended terms.

As used herein, “and/or” refers to and encompasses any and all possible combinations of one or more of the associated listed items.

As used herein, the term “about” or “approximately” refers to within an acceptable error range for the particular value as determined by one of ordinary skill in the art, which will depend in part on how the value is measured or determined, i.e., the limitations of the measurement system. The term “about” can mean a range of up to 5%, up to 1%, up to 0.5%, or even up to 0.1% of a given value. Alternatively, the term “about,” particularly when used with respect to biological systems or processes, can mean within an order of magnitude, or within 2-fold, of a value.

As used herein, the term “consist essentially” means that the ingredients include only the listed components along with the normal impurities present in commercial materials and with any other additives present at levels which do not affect the operation of the disclosure, for instance at levels less than 5% by weight or less than 1% or even 0.5% by weight.

As used herein, the term “pharmaceutical composition” refers to a composition containing one or more antihypertensive drugs (e.g., selected from the group comprising angiotensin converting enzyme inhibitor (ACEi), angiotensin receptor blocker (ARB), diuretic, β-blocker, calcium channel blocker, central α-agonist, peripheral α-antagonist, direct vasodilator, neprilysin inhibitor, and mixtures thereof) described herein formulated with a pharmaceutically acceptable excipient.

As used herein, the term “aqueous” refers to a composition that contains water as a solvent or medium.

The term “degradable,” as used herein, refers to capable of being chemically and/or physically modified, dissolved, or broken down, e.g., in the body of a patient, within the relevant time period.

The term “substantially free,” as used herein, refers to excluding any functional amount, which refers to any amount that contributes or has an effect on the release profile and/or the receptor-binding affinity to of the composition.

The term “high blood pressure,” as used herein, refers to blood pressure levels of 140 mm Hg/90 mm Hg or higher.

The term “normal blood pressure,” as used herein, refers to blood pressure levels of 120 mm Hg/80 mm Hg or lower.

The term “at risk blood pressure,” as used herein, refers to blood pressure levels of 120-139 mmHg/80-89 mm Hg.

The term “dysphagia”, as used herein, refers to difficulty in swallowing.

The term “geriatric”, as used herein, refers to persons from age 65 and older.

The term “pediatric”, as used herein, refers to patients ages from birth to younger than 17 years old.

The terms “dysyphagic patient” and “patient with dysphagia” as used interchangeably herein, refer to patients who have difficulty in swallowing due to cancer, stroke, gastric reflux disease (GERD), chronic obstructive pulmonary disease (COPD), Parkinson's disease, multiple sclerosis, dementia, motor neuron disease, and side effects of commonly prescribed drugs such as antiarrhythmics, antihistamines, decongestants, diuretics, selective serotonin reuptake inhibitors.

The term “hypertensive urgency, as used herein, refers to a condition where the blood pressure is severely elevated (systolic>200 mmHg or diastolic>120 mmHg) with the absence of acute target organ disease.

The term “hypertensive emergency,” as used herein, refers to a condition in which there is an acute, marked elevation of both systolic and diastolic blood pressure resulting in acute target organ disease. Such target organ disease can include pulmonary edema, cardiac ischemia, neurologic deficits, acute renal failure, aortic dissection, eclampsia, and any unknown reason.

The term “hypertensive crisis,” as used herein, refers to a condition wherein there is a sudden development of severely high blood pressure (e.g., 180/120 mmHg or higher).

The terms “essential hypertension,” “primary hypertension,” and ‘idiopathic hypertension,” as used interchangeably herein, refer to high blood pressure in which secondary causes such as renovascular disease, renal failure, pheochromocytoma, aldosteronism, or other causes of secondary hypertension or mendelian forms (monogenic) are not present

The term “subject,” as used herein, refers to any organism to which a composition and/or compound in accordance with the disclosure may be administered, e.g., for experimental, diagnostic, prophylactic, and/or therapeutic purposes. Typical subjects include any animal (e.g., mice, rats, rabbits, non-human primates, and humans, etc.). A subject in need thereof is typically a subject for whom it is desirable to treat a disease, disorder, or condition as described herein. For example, a subject in need thereof may seek or be in need of treatment, require treatment, be receiving treatment, may be receiving treatment in the future, or a human or animal that is under care by a trained professional for a particular disease, disorder, or condition.

The terms “unit dosage forms,” and “unitary dosage forms,” as used interchangeably herein refer to forms of medication supplied in a manner that does not require further weighing or measuring to provide the dosage (e.g., nasal spray, nasal drops, nasal dry powder, nasal pressurized aerosol).

The term “effective amount” or “therapeutically effective amount” of one or more agents (e.g., antihypertensive drugs), as used herein, is the amount sufficient to effect beneficial or desired results, including prophylactic results and/or clinical results. An “effective amount” depends upon the context in which it is being applied. In some embodiments, the compounds are administered in an effective amount for the treatment or prophylaxis of a disease disorder or condition (e.g., high blood pressure). In another embodiment, an effective amount of an agent is, for example, an amount sufficient to achieve alleviation or amelioration or prevention or prophylaxis of one or more symptoms or conditions; diminishment of extent of disease, disorder, or condition; stabilized (i.e., not worsening) state of disease, disorder, or condition; preventing spread of disease, disorder, or condition; delay or slowing the progress of the disease, disorder, or condition; or amelioration or palliation of the disease, disorder, or condition (e.g., high blood pressure, etc.), whether detectable or undetectable, as compared to the response obtained without administration of the agent.

Pharmaceutically effective regimens are typically systematic plans for the administration of one or more therapeutic agents, which includes aspects, e.g., type of therapeutic agent, therapeutic agent concentrations, and any changes therein made during the course of the drug administration, which when administered is capable of (e.g., is effective in, etc.) treating and/or preventing a therapeutic disorder. Pharmaceutically effective regimens may include daily administration or a maximum administration for a period of time (e.g., more than one week such as one week, two weeks, three, weeks, four weeks, etc.). For example, the composition may be administered one or more times daily for several weeks.

The term “liquid compositions,” as used herein, refers to solutions, emulsions or suspensions containing one or more active ingredients (antihypertensive drugs) in a suitable vehicle. The selection of vehicle is based on the nature of the active ingredient(s).

Unless otherwise indicated, all references to concentrations include the indicated amounts on a weight by weight, weight by volume or volume by volume basis. Any reference to a percent concentration will be understood to refer to one of wt/wt, wt/vol, or vol/vol unless otherwise indicated. While certain embodiments may be described by concentrations as wt/wt or wt/vol, it should be understood that some compositions may have the same % on a wt/wt and vol/vol basis.

### Dosage Forms

The present disclosure provides non-parenteral, reliable and convenient dosage forms for antihypertensive drugs using administrative routes where a dissolved or partially dissolved drug is rapidly absorbed into systemic circulation. The present disclosure provides novel nasal compositions comprising antihypertensive drugs. In certain embodiments, the nasal compositions of the disclosure comprise nasal spray compositions, nasal drop compositions, nasal dry powder compositions, and nasal pressurized aerosols. Nasal administration is an efficient way of transporting antihypertensive drugs through nostrils and nasal cavities with potential use in reducing and maintaining blood pressure. Nasal sprays, nasal powders and nasal pressurized aerosols are preferred over nasal drops as they can deliver drugs deeper into nasal cavity where the potential of systemic absorption is high.

In certain embodiments, nasal compositions comprise nasal solutions, nasal suspensions, or nasal emulsions comprising at least one mucoadhesive thickening agent for mucoadhesion and thickening of the composition. In certain embodiments, the viscosity of nasal solution, suspension, or emulsion is an advantage for local application. In certain embodiments, a mucoadhesive solution, suspension, or emulsion provides longer residence time in nasal mucosa, facilitating rapid and complete absorption of drugs. In certain embodiments, the mucoadhesive and thickening agent decreases or prevents dripping of the composition from the nose.

In certain embodiments, the antihypertensive drug is selected from the group comprising angiotensin converting enzyme inhibitor (ACEi), angiotensin receptor blocker (ARB), diuretic, β-blocker, calcium channel blocker, central α-agonist, peripheral α-antagonist, direct vasodilator, neprilysin inhibitor, and mixtures thereof.

In certain embodiments, the angiotensin converting enzyme inhibitor is selected from the group consisting of benazepril, captopril, enalapril, fosinopril, lisinopril, moexipril, perindopril, quinapril, ramipril, trandolapril, alacepril, cilazapril, delapril, imidapril, spirapril, temocapril, zofenopril, and pharmaceutically acceptable salts and forms thereof.

In certain embodiments, the angiotensin receptor blocker is selected from the group consisting of azilsartan, candesartan, eprosartan, irbesartan, losartan, olmesartan, telmisartan, valsartan, fimasartan, and pharmaceutically acceptable salts and forms thereof.

In certain embodiments, the diuretic is selected from the group comprising hydrochlorothiazide, chlorthalidone, furosemide, spironolactone, triamterene, and amiloride. In certain embodiments, diuretics are suitable for administration in adult population. In certain embodiments, diuretics are suitable for administration in pediatric population. In certain embodiments, diuretic used is hydrochlorothiazide.

In certain embodiments, the B-blocker is selected from the group comprising metoprolol succinate, atenolol, propranolol, labetalol, bisoprolol, timolol maleate and mixtures thereof. In certain embodiments, β-blockers are suitable for administration in adult population. In certain embodiments, β-blockers are suitable for administration in pediatric population. In certain embodiments, β-blocker used is propranolol or S(-)-propranolol.

In certain embodiments, the calcium channel blocker is selected from the group comprising diltiazem, nisoldipine, amlodipine, nifedipine, felodipine, nimodipine, isradipine, and mixtures thereof. In certain embodiments, calcium channel blockers are suitable for administration in adult population. In certain embodiments, calcium channel blockers are suitable for administration in pediatric population. In certain embodiments, calcium channel blocker used is amlodipine or S-amlodipine.

In certain embodiments, the central α-agonist is clonidine or lofexidine. In certain embodiments, central α-agonists are suitable for administration in adult population. In certain embodiments, central α-agonists are suitable for administration in pediatric population.

In certain embodiments, the peripheral α-antagonist is selected from the group comprising doxazosin, prazosin, terazosin, and mixtures thereof. In certain embodiments, peripheral α-antagonists are suitable for administration in adult population. In certain embodiments peripheral α-antagonists are suitable for administration in pediatric population.

In certain embodiments, the direct vasodilator used is hydralazine.

In certain embodiments, neprilysin inhibitor used is sacubitril.

In certain embodiments, the compositions of the disclosure are useful for treatment and/or prevention of a condition or a disease selected from the group consisting of hypertension, heart failure, left ventricular dysfunction and hypertrophic cardiomyopathy, diabetic cardiac myopathy, supraventricular and ventricular arrhythmias, atrial fibrillation, atrial flutter, detrimental vascular remodeling, myocardial infarction and its sequelae, atherosclerosis, angina (whether unstable or stable), renal insufficiency (diabetic and non-diabetic), heart failure, angina pectoris, secondary aldosteronism, primary and secondary pulmonary hypertension, renal failure conditions, diabetic retinopathy, the management of other vascular disorders, selected from migraine, peripheral vascular disease, Raynaud's disease, luminal hyperplasia, cognitive dysfunction, glaucoma and stroke.

In certain embodiments, the compositions of the disclosure are suitable for once daily administration for lowering and/or maintaining systemic blood pressure to treat conditions or diseases associated with high blood pressure. In certain embodiments, the compositions of the disclosure are suitable for multiple daily administrations, for lowering and/or maintaining systemic blood pressure. In certain embodiments, the compositions of the disclosure are suitable for at least once daily administration, twice daily administration, thrice daily administration, or are suitable for four daily administrations.

### Nasal Compositions

The nasal compositions of the disclosure comprise nasal solutions, nasal powders, nasal suspensions or nasal emulsions comprising an antihypertensive drug.

In certain embodiments, the nasal compositions of the disclosure comprise an antihypertensive drug and a buffer to maintain the pH of the solution from about 6 to about 7. In certain embodiments, the nasal composition exhibits a pH about 6, about 6.1, about 6.2, about 6.3, about 6.4, about 6.5, about 6.6, about 6.7, about 6.8, about 6.9, about 7, or any intermediate values therein. In certain embodiments, the pH of nasal composition of the disclosure is from about 6.2 to about 6.6.

In certain embodiments, the nasal compositions comprising nasal solutions, suspensions, or emulsions are administered as nasal sprays.

Nasal sprays are used to deliver drugs/medications locally or systemically. They are generally used locally for conditions such as nasal congestion and allergic rhinitis. The nasal delivery route is preferred for quick absorption and delivery of the medication directly through nose.

Nasal spray compositions of the disclosure comprise aqueous solutions, suspensions, or oil-in-water emulsions for delivering antihypertensive drugs systemically as fine droplets or mists in the nasal cavity.

In certain embodiments, the nasal spray products comprise:
a body configured to be inserted into a nasal passage for dispersing a nasal spray composition through an orifice;
a reservoir in fluid communication with the orifice, wherein the nasal composition is contained in the reservoir;
a pump mechanism capable of expelling the nasal spray composition through the orifice in appropriately sized aerosolized droplets, capable of coating the nasal mucosa of a user, wherein the nasal spray composition comprises an antihypertensive drug.

In certain embodiments the nasal composition is packaged as unit dose or bi-dose vials to keep the composition sterile and avoid the invasion of microbial agents. In certain embodiments the nasal composition is packaged as multidose vials with a cartridge pump system (CPS) containing a 0.2 μm filter to keep the composition sterile and avoid the invasion of microbial agents.

In certain embodiments the nasal composition containing a preservative to avoid microbial growth is packaged as multidose vials.

Delivery through nasal spray improves patient compliance and convenience by providing quick absorption of doses in a short period of time. Additionally, nasal spray provides advantages of drug delivery as fine droplets or mist in the nasal cavity, which results in rapid onset of a therapeutic effect, reduced required drug dose, and reduced systemic side effects. In certain embodiments, the nasal sprayer is used together with a nasal spray bottle and a nasal spray pump to deliver the nasal composition in each nostril. In certain embodiments, by pressing the nasal pump, the nasal spray composition is sprayed as fine droplets or mist into the nasal cavity. In certain embodiments, nasal spray pump is mechanically pressed to release the nasal spray composition as fine droplets or mist. In certain embodiments, the nasal spray pump is pressed electrically to release nasal spray composition as fine droplets or mist.

In certain embodiments, the nasal spray application is designed in a way that an optimal size distribution is targeted at a desired dosing volume. In certain embodiments, the optimal droplet size distribution is from about 2 μm to about 120 μm, from about 5 μm to about 115 μm, from about 10 μm to about 110 μm, from about 15 to 100 μm, from about 20 μm to about 90 μm, from about 25 μm to about 80 μm, from about 30 μm to about 70 μm, from about 35 μm to about 60 μm, from about 40 μm to about 50 μm, or intermediate values therein. In certain embodiments, the desired dosing volume/spray is from about 1 μl to about 200 μl. In certain embodiments, the desired dosing volume/spray is about 10 μl, about 20 μl, about 30 μl, about 40 μl, about 50 μl, about 60 ml, about 70 μl, about 80 μl, about 90 μl, about 100 μl, about 110 μl, about 120 μl, about 130 μl, about 140 μl, about 150 μl, about 160 μl, about 170 μl, about 180 μl, about 190 μl, about 200 μl, or any intermediate volumes therein. In certain embodiments, the desired dosing volume/spray is about 100 μl. In certain embodiments, with the use of metered-dose pumps and actuators, the nasal spray delivers a dose of from about 20 μl to about 200 μl/spray. In certain embodiments, with the use of metered-dose pumps and actuators, the nasal spray delivers a dose of about 100 μl/spray. In certain embodiments, the particle size and morphology (in the suspension) of the drug and viscosity of the composition determine the choice of pump and actuator assembly.

In certain embodiments, the nasal compositions of the disclosure comprising an antihypertensive drug are administered as nasal spray compositions providing faster systemic drug absorption compared to an oral solution of the same drug. In certain embodiments, the nasal spray composition of the disclosure comprising an antihypertensive drug provides a post-dose lag time to systemic drug absorption that is at least about 30 minutes less than the post-dose lag time to systemic drug absorption from an oral dosage form (e.g., oral tablet or solution) containing the same drug. In certain embodiments, the nasal spray composition of the disclosure comprising an antihypertensive drug provides a post-dose lag time to systemic drug absorption that is from about 30 minutes to about 2 hours less than the post-dose lag time to the systemic drug absorption from an oral dosage form (e.g., oral tablet or solution) containing the same drug. In certain embodiments, nasal spray composition of the disclosure comprising an antihypertensive drug provides a post-dose lag time to systemic drug absorption that is from about 30 minutes to about 60 minutes less than the post-dose lag time to systemic drug absorption from an oral dosage form (e.g., oral tablet or solution) containing the same drug. In certain embodiments, the nasal spray composition of the disclosure comprising an antihypertensive drug provides a post-dose lag time to systemic drug absorption that is about 30 minutes less, about 35 minutes less, about 40 minutes less, about 45 minutes less, about 50 minutes less, about 55 minutes less, about 60 minutes less, or any intermediate periods therein, than the lag time to the systemic drug absorption from oral dosage form (e.g., oral tablet or solution) containing the same drug.

In certain embodiments, systemic drug (antihypertensive drug) absorption provided by the nasal compositions of the disclosure at about 5 minutes post-dose is at least similar to the systemic drug absorption from a marketed oral solution containing the same drug at about 1.5 hours to about 2 hours post-dose. In certain embodiments, the nasal composition of the disclosure, when dose normalized to a marketed oral solution containing the same drug, provides at least 2-fold increase in drug plasma concentration at about 1.5 hours post-dose compared to 1.5 hours post-dose of the marketed oral solution containing the same drug. In certain embodiments, the nasal composition of the disclosure, when dose normalized to a marketed oral solution containing the same antihypertensive drug, provides about 2-fold, about 3-fold, about 4-fold, about 5-fold, about 6-fold, about 7-fold, about 8-fold, about 9-fold increase, or any intermediate values therein of drug plasma concentration at about 1.5 hours post-dose compared to the marketed oral solution containing the same drug.

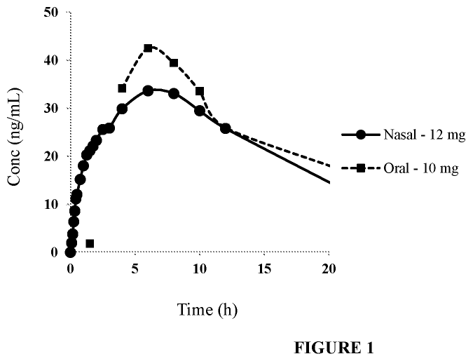

FIG. 1/Table 2 compare systemic bioavailability of 12 mg lisinopril equivalent dose of aqueous test composition, administered as nasal spray comprising 6 mg lisinopril equivalent/100 μL/nostril in each of the two nostrils, to an equivalent oral dose of marketed lisinopril product (literature data for 10 mL marketed lisinopril oral solution (1 mg/mL)), when administered under fasting conditions.

Data from FIG. 1/Table 2 clearly demonstrates that aqueous lisinopril nasal spray composition provides faster systemic absorption compared to marketed lisinopril oral solution. The aqueous lisinopril composition administered as nasal spray provides a post-dose lag time to systemic drug absorption that is about 1.5 hours less than the lag time to the systemic drug absorption from marketed lisinopril oral solution. Data from Table 2 shows about 9-fold increase in lisinopril plasma concentration from the aqueous lisinopril nasal spray composition of the disclosure compared to lisinopril plasma concentration from marketed oral lisinopril solution at about 1.5 hours post-dose.

Table 3 provides Cₘₐₓ, Tₘₐₛ, AUC (0-24), AUC (0-∞), and T_{1/2} pharmacokinetic parameters for the aqueous lisinopril nasal spray composition of the disclosure.

In certain embodiments, the nasal antihypertensive compositions (e.g., compositions comprising an antihypertensive drug) of the disclosure comprising nasal solutions, suspensions, or emulsions are administered as nasal drops. Nasal drops are one of the most simple and convenient nasal delivery systems for delivering drug/medication directly into the nasal cavity. Drops are widely spread through nasal cavity and reach the posterior of the nasal cavity, where mucociliary clearance is more active, creating rapid clearance from the cavity.

In certain embodiments, a nasal dropper is used to deliver nasal compositions in each nostril as nasal drops. In certain embodiments, the nasal drops deliver a therapeutic dose of desired antihypertensive drug in the form of droplets. The drops are administered by sucking liquid into a glass or plastic dropper, inserting the dropper into the nostril with an extended neck before squeezing the rubber top to emit the drops. In certain embodiments, in patients with nasal polyps, nasal drop composition is preferred over nasal sprays. Although drops work well for some patients, their popularity is limited by the need for head-down body positions and/or extreme neck extension required for the desired gravity-driven deposition of drops.

In certain embodiments, the nasal compositions of the disclosure comprising an antihypertensive drug are dry powder compositions suitable for nasal administration via powder exhalation delivery system (EDS). In certain embodiments, the dry powder compositions are supplied in medication capsules pre-filled in a nosepiece section of the EDS. Such delivery systems can be used to deliver the antihypertensive drug via nose using a reusable device body incorporating a flexible mouth piece to adjust individual anatomic variations, and a piercing assembly to pierce the medication capsule. In certain embodiments, the powder is delivered using a device with a reservoir (e.g., turbuhaler).

In certain embodiments, nasal suspension compositions of the disclosure comprising an antihypertensive drug are delivered as nasal pressurized metered aerosols comprising micronized or nanosized antihypertensive drug.

In certain embodiments, the antihypertensive drug selected from the group comprising angiotensin converting enzyme inhibitor (ACEi), angiotensin receptor blocker (ARB), diuretic, β-blocker, calcium channel blocker, central α-agonist, peripheral α-antagonist, direct vasodilator, neprilysin inhibitor, and mixtures thereof.

In certain embodiments, the antihypertensive drug has a D50 particle size of less than or equal to about 5 μm, e.g., from about 1 μm to about 5 μm. In certain embodiments, the drug has a D50 particle size of from about 2 μm to about 8 μm, e.g., about 2 μm, about 2.5 μm, about 3 μm, about 3.5 μm, about 4 μm, about 4.5 μm, about 5 μm, about 5.5 μm, about 6 μm, about 6.5 μm, about 7 μm, about 7.5 μm, about 8 μm, or any intermediate values therein.

### Nasal Solutions

In certain embodiments, the nasal compositions of the disclosure are aqueous nasal solutions comprising an antihypertensive drug and a buffer. In certain embodiments, the aqueous nasal solutions comprise an antihypertensive drug (nasal antihypertensive solution), a buffer, and optionally, a mucoadhesive thickening agent, a preservative, a tonicity agent, a penetration enhancer, and/or a humectant. In certain embodiments, the nasal solutions are administered as nasal spray or nasal drops.

In certain embodiments, the antihypertensive drug is selected from the group comprising angiotensin converting enzyme inhibitor (ACEi), angiotensin receptor blocker (ARB), diuretic, β-blocker, calcium channel blocker, central α-agonist, peripheral α-antagonist, direct vasodilator, neprilysin inhibitor, and mixtures thereof.

In certain embodiments, the buffer is selected from the group comprising sodium dihydrogen phosphate; disodium hydrogen phosphate; sodium citrate and citric acid; sodium acetate, acetic acid; maleic acid and salts; carbonic acid, ammonia, carbonate, sodium carbonate, decarbonate sodium, trisodium citrate, lactic acid, maleic acid, tartaric acid, sodium tartrate and salts, glycine, tris (hydroxymethyl) aminomethane, and mixtures thereof.

In certain embodiments, the mucoadhesive thickening agent is selected from the group comprising carboxymethylcellulose, sodium carboxymethylcellulose, methyl cellulose, hydroxypropyl methylcellulose, hydroxypropyl cellulose, hydroxyethyl cellulose, microcrystalline cellulose, mixture of microcrystalline cellulose and carboxymethylcellulose sodium, poloxamer, polyvinyl alcohol, polyvinyl pyrrolidone, polyvinyl acetate, polydextrose, pectin, chitosan, acacia, polyethylene glycol, sodium alginate, bentonite, carbomer, carrageenan and, guar gum, alginic acid, carbomer, hectorite, povidone, tragacanth, xanthan gum and mixtures thereof. In certain embodiments, mucoadhesive agent is present in an amount of up to about 5% wt/vol of the nasal antihypertensive solution. In certain embodiments, mucoadhesive agent is present in an amount of about 0.1% wt/vol, about 0.5% wt/vol, about 1% wt/vol, about 1.5% wt/vol, about 2% wt/vol, about 2.5% wt/vol, about 3% wt/vol, about 3.5% wt/vol, about 4% wt/vol, about 4.5% wt/vol, about 5% wt/vol, or intermediate values therein of the nasal antihypertensive solution.

In certain embodiments, the preservative is added to prevent or minimize microbial growth in the composition. In certain embodiments, the preservative is selected from the group comprising benzalkonium chloride (BAC), potassium sorbate, methyl paraben, propyl paraben, benzyl alcohol, benzoic acid, phenoxyethanol, chlorobutanol, m-cresol, phenol, thiomersal, and mixtures thereof.

In certain embodiments, the nasal antihypertensive solution is free of any preservative. In certain embodiments, the preservative is present in an amount of up to about 3% wt/vol of the nasal antihypertensive solution. In certain embodiments, the preservative is present in an amount of about 0.01% wt/vol, about 0.02% wt/vol, about 0.03% wt/vol, about 0.04% wt/vol, about 0.05% wt/vol, about 0.06% wt/vol, about 0.07% wt/vol, about 0.08% wt/vol, about 0.09% wt/vol, about 0.1% wt/vol, about 0.2% wt/vol, about 0.5% wt/vol, about 1% wt/vol, about 1.5% wt/vol, about 2% wt/vol, about 2.5% wt/vol, about 3% wt/vol or intermediate values therein of the nasal antihypertensive solution.

In certain embodiments, the nasal antihypertensive solution compositions of the disclosure further comprise ethylenediamine tetraacetic acid (EDTA) as a chelating agent. In certain embodiments, ethylenediamine tetraacetic acid (EDTA) and salts thereof is used in combination with antimicrobial preservatives to enhance their activity.

In certain embodiments, the solubility of the antihypertensive drug is improved by addition of a co-solvent selected from the group comprising ethanol, polyethylene glycol, triacetin, propylene glycol, glycerin, polypropylene glycol, sorbitol and mixtures thereof.

In certain embodiments, the humectant is selected from the group comprising glycerol, propylene glycol, sorbitol, triacetin, mannitol, sucrose, glucose, allantoin, and mixtures thereof. In certain embodiment, the humectant can be glycerol. In certain embodiments, humectant is present in an amount of up to about 20% wt/vol of the nasal antihypertensive solution. In certain embodiments, humectant is present in an amount of about 0.5% wt/vol, about 1% wt/vol, about 1.5% wt/vol, about 2% wt/vol, about 2.5% wt/vol, about 5% wt/vol, about 10% wt/vol, about 15% wt/vol, about 20% wt/vol, or intermediate values therein of the nasal antihypertensive solution.

In certain embodiments, the penetration enhancer is selected from the group comprising glycosides, benzalkonium chloride, benzethonium chloride, cetylpyridinium chloride, cyclodextrin, glycine, lecithin, saponins, soybean stearyl glucosides, sucrose esters of fatty acids, surfactant (polyoxyethylene-9-lauryl ether, polysorbate 80, sodium deoxycholate, sodium taurocholate, and mixtures thereof.

In certain embodiments, the nasal antihypertensive solution further comprises at least one osmotic agent/tonicity agent to adjust tonicity of the composition with body's natural fluids. In certain embodiments, the osmotic agent is selected from the group comprising sodium chloride, dextrose, glycerin, potassium chloride, mannitol, calcium chloride, glucose, glycine, magnesium chloride, potassium chloride, sorbitol, sucrose, sodium sulfate, and mixtures thereof. In certain embodiments, tonicity agent is present in an amount of up to about 7% wt/vol of the nasal antihypertensive solution. In certain embodiments, tonicity agent is present in an amount of about 0.1% wt/vol, about 0.5% wt/vol, about 1% wt/vol, about 2% wt/vol, about 3% wt/vol, about 4% wt/vol, about 5% wt/vol, about 6% wt/vol, about 7% wt/vol, or intermediate values therein of the nasal antihypertensive solution.

In certain embodiments, the nasal antihypertensive solution further comprises at least one pH adjusting agent to adjust the pH to physiological conditions and improve stability of the active agent. In certain embodiments, the pH adjusting agent is selected from the group comprising hydrochloric acid, sulfuric acid, and sodium hydroxide. In certain embodiments, the pH of the nasal solution is from about 4 to about 7.5. In certain embodiments, the pH of the nasal solution is about 4, about 4.1, about 4.2, about 4.3, about 4.4, about 4.5, about 4.6, about 4.7, about 4.8, about 4.9, about 5, about 5.1, about 5.2, about 5.3, about 5.4, about 5.5, about 5.6, about 5.7, about 5.8, about 5.9, about 6, about 6.1, about 6.2, about 6.3, about 6.4, about 6.5, about 6.6, about 6.7, about 6.8, about 6.9, about 7, about 7.1, about 7.2, about 7.3, about 7.4, about 7.5, or any intermediate values therein.

In certain embodiments, the nasal antihypertensive solution is a nasal lisinopril solution comprising from about 4 mg/100 μl to about 8 mg/100 μl lisinopril (e.g., about 6 mg/100 μl lisinopril) and a buffer. In certain embodiments, the nasal lisinopril solution comprises lisinopril salt or hydrate thereof (e.g., about 6.5 mg/100 μl lisinopril dihydrate), a buffer, and optionally, a mucoadhesive thickening agent, a preservative, a tonicity agent, a penetration enhancer, and/or a humectant. In certain embodiments, the nasal lisinopril solution is preservative free.

In certain embodiments, the nasal antihypertensive solutions of the disclosure are manufactured by adding required ingredients with mixing in the following order: about 60% of sterile water for injection, weak acid, antihypertensive drug (e.g., angiotensin converting enzyme inhibitor), thickening agent, humectant, salt of the weak acid, and remaining water for injection. The final clear solution is filtered aseptically and stored in an acceptable holding vessel. In some embodiments the final clear solution is filtered and stored in a sterile holding vessel under nitrogen blanket, if necessary, until it is filled into bottles/vials and capped with spray/pump device.

### Nasal Dry Powder Compositions

In certain embodiments, the nasal antihypertensive compositions of the disclosure are dry powder compositions suitable for nasal administration via powder exhalation delivery system (EDS). In certain embodiments, the dry powder compositions are supplied in medication capsules pre-filled with an antihypertensive drug in a nosepiece section of the EDS. Such delivery systems can be used to deliver the antihypertensive drug via nose using a reusable device body incorporating a flexible mouth piece to adjust individual anatomic variations, and a piercing assembly to pierce the medication capsule. In certain embodiments, the powder comprising an antihypertensive drug is delivered using a device with a reservoir (e.g., turbuhaler).

In certain embodiments, the antihypertensive drug is selected from the group comprising angiotensin converting enzyme inhibitor (ACEi), angiotensin receptor blocker (ARB), diuretic, β-blocker, calcium channel blocker, central α-agonist, peripheral α-antagonist, direct vasodilator, neprilysin inhibitor, and mixtures thereof.

The dry powder compositions are manufactured by micronizing or nanonizing the antihypertensive drug, filling the micronized or nanonized drug into a gelatin or hydroxypropylmethylcellulose (HPMC) or starch or pullulan capsule for the powder exhalation delivery system, or a reservoir of a turbuhaler device or other nasal powder delivery devices. Nasal Suspensions

In certain embodiments, nasal antihypertensive compositions of the disclosure are nasal suspensions comprising an antihypertensive drug, a buffer, a surfactant, a suspending agent and optionally, a preservative, a humectant, a penetration enhancer, and/or a tonicity agent.

In certain embodiments, the nasal antihypertensive suspensions are administered as nasal spray, nasal drops, or pressurized metered aerosols.

In certain embodiments, the antihypertensive drug is selected from the group comprising angiotensin converting enzyme inhibitor (ACEi), angiotensin receptor blocker (ARB), diuretic, β-blocker, calcium channel blocker, central α-agonist, peripheral α-antagonist, direct vasodilator, neprilysin inhibitor, and mixtures thereof.

In certain embodiments, the buffer is selected from the group comprising sodium dihydrogen phosphate; disodium hydrogen phosphate; sodium citrate and citric acid; sodium acetate, acetic acid; maleic acid and salts; carbonic acid, ammonia, carbonate, sodium carbonate, decarbonate sodium, trisodium citrate, lactic acid, maleic acid, tartaric acid, sodium tartrate and salts, glycine, tris (hydroxymethyl) aminomethane, and mixtures thereof.

In certain embodiments, the suspending agent is selected from the group comprising carboxymethylcellulose, sodium carboxymethylcellulose, methyl cellulose, hydroxypropyl methylcellulose, hydroxypropyl cellulose, hydroxyethyl cellulose, microcrystalline cellulose, mixture of microcrystalline cellulose and carboxymethylcellulose sodium, poloxamer, polyvinyl alcohol, polyvinyl pyrrolidone, polyvinyl acetate, polydextrose, pectin, chitosan, acacia, polyethylene glycol, sodium alginate, bentonite, carbomer, carrageenan and, guar gum, alginic acid, carbomer, hectorite, povidone, tragacanth, xanthan gum and mixtures thereof. In certain embodiments, the suspending agent is present in an amount of up to about 5% wt/vol of the nasal antihypertensive suspension. In certain embodiments, suspending agent is present in an amount of about 0.1% wt/vol, about 0.5% wt/vol, about 1% wt/vol, about 1.5% wt/vol, about 2% wt/vol, about 2.5% wt/vol, about 3% wt/vol, about 3.5% wt/vol, about 4% wt/vol, about 4.5% wt/vol, about 5% wt/vol, or intermediate values therein of the nasal antihypertensive suspension.

In certain embodiments, the preservative is selected from the group comprising benzalkonium chloride (BAC), potassium sorbate, methyl paraben, propyl paraben, benzyl alcohol, benzoic acid, phenoxyethanol, chlorobutanol, m-cresol, phenol, thiomersal, and mixtures thereof. In certain embodiments, the solution is free of any preservative. In certain embodiments, the preservative is present in an amount of up to about 3% wt/vol of the nasal antihypertensive suspension. In certain embodiments, the preservative is present in an amount of about 0.01% wt/vol, about 0.05% wt/vol, about 0.1% wt/vol, about 0.2% wt/vol, about 0.5% wt/vol, about 1% wt/vol, about 1.5% wt/vol, about 2% wt/vol, about 2.5% wt/vol, about 3% wt/vol or intermediate values therein of the nasal antihypertensive suspension.

In certain embodiments, the nasal antihypertensive suspension compositions of the disclosure further comprise ethylenediamine tetraacetic acid (EDTA) as a chelating agent. In certain embodiments, ethylenediamine tetraacetic acid (EDTA) and salts thereof is used in combination with antimicrobial preservatives to enhance their activity.

In certain embodiments, the solubility of the antihypertensive drug is improved by addition of a co-solvent selected from the group comprising ethanol, polyethylene glycol, triacetin, propylene glycol, glycerin, polypropylene glycol, sorbitol and mixtures thereof.

In certain embodiments, the humectant is selected from the group comprising glycerol, propylene glycol, sorbitol, triacetin, mannitol, sucrose, glucose, allantoin, and mixtures thereof. In certain embodiment, the humectant can be glycerol. In certain embodiments, humectant is present in an amount of up to about 20% wt/vol of the nasal antihypertensive suspension. In certain embodiments, humectant is present in an amount of about 0.5% wt/vol, about 1% wt/vol, about 1.5% wt/vol, about 2% wt/vol, about 2.5% wt/vol, about 5% wt/vol, about 10% wt/vol, about 15% wt/vol, about 20% wt/vol, or intermediate values therein of the nasal antihypertensive suspension.

In certain embodiments, the penetration enhancer is selected from the group comprising glycosides, benzalkonium chloride, benzethonium chloride, cetylpyridinium chloride, cyclodextrin, glycine, lecithin, saponins, soybean stearyl glucosides, sucrose esters of fatty acids, surfactant (polyoxyethylene-9-lauryl ether, polysorbate 80, sodium deoxycholate, sodium taurocholate, and mixtures thereof.

In certain embodiments, the tonicity agent is selected from the group comprising sodium chloride, dextrose, glycerin, potassium chloride, mannitol, calcium chloride, glucose, glycine, magnesium chloride, potassium chloride, sorbitol, sucrose, sodium sulfate, and mixtures thereof.

In certain embodiments, tonicity agent is present in an amount of up to about 7% wt/vol of the nasal antihypertensive suspension. In certain embodiments, tonicity agent is present in an amount of about 0.1% wt/vol, about 0.5% wt/vol, about 1% wt/vol, about 2% wt/vol, about 3% wt/vol, about 4% wt/vol, about 5% wt/vol, about 6% wt/vol, about 7% wt/vol, or intermediate values therein of the nasal antihypertensive suspension.

In certain embodiments, the pH adjusting agent is selected from the group comprising hydrochloric acid, sulfuric acid, and sodium hydroxide. In certain embodiments, the pH of the nasal suspension is from about 4 to about 7.5. In certain embodiments, the pH of the nasal suspension is about 4, about 4.1, about 4.2, about 4.3, about 4.4, about 4.5, about 4.6, about 4.7, about 4.8, about 4.9, about 5, about 5.1, about 5.2, about 5.3, about 5.4, about 5.5, about 5.6, about 5.7, about 5.8, about 5.9, about 6, about 6.1, about 6.2, about 6.3, about 6.4, about 6.5, about 6.6, about 6.7, about 6.8, about 6.9, about 7, about 7.1, about 7.2, about 7.3, about 7.4, about 7.5, or any intermediate values therein.

In certain embodiments, presence of a surfactant increases stability and suspendability of the suspension. In certain embodiments, the surfactant is selected from the group comprising polysorbate polysorbate 20 (polyoxyethylene (20) sorbitan monooleate), polysorbate 80/Tween (polyoxyethylene (80) sorbitan monooleate), dipalmitoylphosphatidylcholine (DPCC), lecithin, oleic acid, sorbitan esters, ammonium lauryl sulfate, trolamine lauryl sulfate, polysorbate 60, other nonionic or ionic surfactants, and mixtures thereof. In certain embodiments, surfactant is present in an amount of up to about 10% wt/vol of the nasal antihypertensive suspension. In certain embodiments, surfactant is present in an amount of about 0.01% wt/vol, about 0.05% wt/vol, about 1% wt/vol, about 2% wt/vol, about 3% wt/vol, about 4% wt/vol, about 5% wt/vol, about 6% wt/vol, about 7% wt/vol, about 8% wt/vol, about 9% wt/vol, about 10% wt/vol, or intermediate values therein of the nasal antihypertensive suspension.

In certain embodiments, nasal antihypertensive suspensions administered as pressurized metered aerosols further comprise a propellant selected from the group comprising nitrogen, carbon dioxide, air, butane, Hydroxy fluoroalkane (e.g., HFA-134a, HFA-227, HFA-152a), and Hydrofluoroolefin (e.g., HFO 1234ze).

In certain embodiments, nasal antihypertensive suspension compositions of the disclosure are manufactured by adding required ingredients with mixing and homogenization in the following order: about 30% of sterile water for injection, mucoadhesive thickening agent, micronized or nanosized antihypertensive agent, osmotic agent, buffer, preservative, humectant, surfactant, and remaining water for injection. The suspension is stored in a holding vessel. In some embodiments the suspension is stored under nitrogen blanket, if necessary, until filled into appropriate container and delivery device.

### Nasal Emulsions

In certain embodiments, nasal antihypertensive compositions of the disclosure are oil-in-water emulsions comprising an antihypertensive drug, a buffer, an emulsifying agent, and optionally, a pH adjusting agent, a preservative, a humectant, a penetration enhancer, and/or a tonicity agent.

In certain embodiments, the nasal emulsions are administered as nasal spray, or nasal drops.

In certain embodiments, the antihypertensive drug is selected from the group comprising angiotensin converting enzyme inhibitor (ACEi), angiotensin receptor blocker (ARB), diuretic, β-blocker, calcium channel blocker, central α-agonist, peripheral α-antagonist, direct vasodilator, neprilysin inhibitor, and mixtures thereof.

In certain embodiments, the oil is selected from the group comprising medium chain triglycerides (MCT), purified vegetable oils, cetyl, decyl, hexadecyl, isodecyl, lauryl, oleyl, stearyl, and tridecyl alcohols, beeswax, butyl stearate, candelilla wax, castor oil, ceresin wax, corn oil, cottonseed oil, decyl acetate, diisosocetyl phthalate, dimethylsilicone, ethyl aniline, isopropyl stearate, isopropyl palmitate, isosteric acid, lanolin, lauric acid, lauryl amine, linoleic acid, maize oil, methyl silicone, methylphenylsilicone, microcrystalline wax, mineral oil, heavy, mineral oil light, oleic acid, palm oil, paraffin, petrolatum, polyethylene wax, propylene tetramer, rapeseed oil, ricinoleic acid, safflower oil, silicone oil, soybean oil, stearic acid, and mixtures thereof.

In certain embodiments, the buffer is selected from the group comprising sodium dihydrogen phosphate; disodium hydrogen phosphate; sodium citrate and citric acid; sodium acetate, acetic acid; maleic acid and salts; carbonic acid, ammonia, carbonate, sodium carbonate, decarbonate sodium, trisodium citrate, lactic acid, maleic acid, tartaric acid, sodium tartrate and salts, glycine, tris (hydroxymethyl) aminomethane, and mixtures thereof.

In certain embodiments, the emulsifying agent is selected from the group comprising polysorbate polysorbate 20 (polyoxyethylene (20) sorbitan monooleate), polysorbate 80/Tween (polyoxyethylene (80) sorbitan monooleate), dipalmitoylphosphatidylcholine (DPCC), lecithin, oleic acid, sorbitan esters, ammonium lauryl sulfate, trolamine lauryl sulfate, polysorbate 60, and other nonionic or ionic surfactants. In certain embodiments, the emulsifying agent is present in an amount of up to about 10% wt/vol of the nasal antihypertensive emulsion. In certain embodiments, surfactant is present in an amount of about 0.01% wt/vol, about 0.02% wt/vol, about 0.05% wt/vol, about 1% wt/vol, about 0.2% wt/vol, about 5% wt/vol, about 8% wt/vol, about 10% wt/vol, or intermediate values therein of the nasal antihypertensive emulsion.

In certain embodiments, the preservative is selected from the group comprising benzalkonium chloride (BAC), potassium sorbate, methyl paraben, propyl paraben, benzyl alcohol, benzoic acid, phenoxyethanol, chlorobutanol, m-cresol, phenol, thiomersal, and mixtures thereof. In certain embodiments, the solution is free of any preservative. In certain embodiments, preservative is present in an amount of up to about 3% wt/vol of the nasal antihypertensive emulsion. In certain embodiments, the preservative is present in an amount of about 0.01% wt/vol, about 0.05% wt/vol, about 0.1% wt/vol, about 0.2% wt/vol, about 0.5% wt/vol, about 1% wt/vol, about 1.5% wt/vol, about 2% wt/vol, about 2.5% wt/vol, about 3% wt/vol or intermediate values therein of the nasal antihypertensive emulsion.

In certain embodiments, the nasal antihypertensive emulsion compositions of the disclosure further comprise ethylenediamine tetraacetic acid (EDTA) as a chelating agent. In certain embodiments, ethylenediamine tetraacetic acid (EDTA) and salts thereof is used in combination with antimicrobial preservatives to enhance their activity.

In certain embodiments, the solubility of the antihypertensive drug is improved by addition of a co-solvent selected from the group comprising ethanol, polyethylene glycol, triacetin, propylene glycol, glycerin, polypropylene glycol, sorbitol and mixtures thereof.

In certain embodiments, the penetration enhancer is selected from the group comprising glycosides, benzalkonium chloride, benzethonium chloride, cetylpyridinium chloride, cyclodextrin, glycine, lecithin, saponins, soybean stearyl glucosides, sucrose esters of fatty acids, surfactant (polyoxyethylene-9-lauryl ether, polysorbate 80, sodium deoxycholate, sodiumtaurocholate, and mixtures thereof.

In certain embodiments, the tonicity agent is selected from the group comprising sodium chloride, dextrose, glycerin, potassium chloride, mannitol, calcium chloride, glucose, glycine, magnesium chloride, potassium chloride, sorbitol, sucrose, sodium sulfate, and mixtures thereof. In certain embodiments, tonicity agent is present in an amount of up to about 7% wt/vol of the nasal antihypertensive emulsion. In certain embodiments, tonicity agent is present in an amount of about 0.1% wt/vol, about 0.5% wt/vol, about 1% wt/vol, about 2% wt/vol, about 3% wt/vol, about 4% wt/vol, about 5% wt/vol, about 6% wt/vol, about 7% wt/vol, or intermediate values therein of the nasal antihypertensive emulsion.

In certain embodiments, the pH adjusting agent is selected from the group comprising hydrochloric acid, sulfuric acid, and sodium hydroxide. In certain embodiments, the pH of the nasal emulsion is from about 4.0 to about 7.5. In certain embodiments, the pH of the nasal suspension is about 4, about 4.1, about 4.2, about 4.3, about 4.4, about 4.5, about 4.6, about 4.7, about 4.8, about 4.9, about 5, about 5.1, about 5.2, about 5.3, about 5.4, about 5.5, about 5.6, about 5.7, about 5.8, about 5.9, about 6, about 6.1, about 6.2, about 6.3, about 6.4, about 6.5, about 6.6, about 6.7, about 6.8, about 6.9, about 7, about 7.1, about 7.2, about 7.3, about 7.4, about 7.5, or any intermediate values therein.

In certain embodiments, nasal antihypertensive emulsion compositions of the disclosure are manufactured by adding required ingredients with mixing and homogenization in the following order: oil, emulsifying agents, micronized or nanosized antihypertensive drug and about 30% sterile water, osmotic agent, buffer, preservative, and remaining sterile water. The emulsion is stored in an appropriate holding vessel. In some embodiment the emulsion is stored under nitrogen blanket, if necessary, until filled in bottles/vials and capped with spray/pump device.

### Dosing Regimen

In certain embodiments, the nasal antihypertensive compositions of the disclosure are suitable for at least once daily administration for lowering and/or maintaining systemic blood pressure to normal level or acceptable level. In certain embodiments, the nasal antihypertensive compositions of the disclosure are suitable for multiple daily administrations. In certain embodiments, the nasal antihypertensive compositions of the disclosure are suitable for at least once daily administration, twice daily administration, or thrice daily or four daily administrations.

In certain embodiments, the nasal antihypertensive compositions of the disclosure are administered as nasal sprays in a dosing regimen comprising once-a-day administration in one or both nostrils as a single spray, two sprays, three or four sprays/nostril.

In certain embodiments, the nasal antihypertensive compositions of the disclosure are administered as nasal drops in a dosing regimen comprising once-a-day administration in one or both nostrils as a single drop, two drops, three or four drops/nostril.

In certain embodiments, the single spray or drop comprises a dosing volume of from about 1 μl to about 200 μl (e.g., dosing volume of nasal spray or drop solution, suspension, emulsion). In certain embodiments, the desired dosing volume/spray is about 10 μl, about 20 μl, about 30 μl, about 40 μl, about 50 μl, about 60 ml, about 70 μl, about 80 μl, about 90 μl, about 100 μl, about 110 μl, about 120 μl, about 130 μl, about 140 μl, about 150 μl, about 160 μl, about 170 μl, about 180 μl, about 190 μl, about 120 μl, or any intermediate volumes therein. In certain embodiments, the desired dosing volume/spray is about 100 μl.

### Methods of Treatment

In certain embodiments, the disclosure provides for a method for treating hypertension in pediatric patients, adult patients, and geriatric patients, the method comprising administering to the patient a nasal composition comprising an antihypertensive drug (nasal antihypertensive composition).

In certain embodiments, the nasal antihypertensive compositions of the disclosure are suitable for treating hypertension in patients with dysphagia. In certain embodiments, the dysphagia is associated with conditions selected from the group consisting of cancer, stroke, Parkinson's disease, multiple sclerosis, gastroesophageal reflux disease (GERD), chronic obstructive pulmonary disease (COPD), dementia, motor neuron disease, and side effects of commonly prescribed drugs selected from the group consisting of antiarrhythmics, antihistamines, decongestants, diuretics, and serotonin reuptake inhibitors.

In certain embodiments, the disclosure provides a method of treating a patient suffering from hypertension associated with hypertensive crisis, hypertensive emergency, or hypertensive urgency, the method comprising administering to the patient a nasal composition of the disclosure comprising an antihypertensive drug.

In certain embodiments, the disclosure provides a method of treating a patient suffering from myocardial infarction, heart failure or cardiovascular disease (CVD), the method comprising administering to the patient nasal composition of the disclosure comprising an antihypertensive drug.

In certain embodiments, the nasal antihypertensive compositions of the disclosure are useful for treatment and/or prevention of a condition or a disease selected from the group consisting of hypertension, heart failure, left ventricular dysfunction and hypertrophic cardiomyopathy, diabetic cardiac myopathy, supraventricular and ventricular arrhythmias, atrial fibrillation, atrial flutter, detrimental vascular remodeling, myocardial infarction and its sequelae, atherosclerosis, angina (whether unstable or stable), renal insufficiency (diabetic and non-diabetic), heart failure, angina pectoris, secondary aldosteronism, primary and secondary pulmonary hypertension, renal failure conditions, diabetic retinopathy, the management of other vascular disorders, selected from migraine, peripheral vascular disease, Raynaud's disease, luminal hyperplasia, cognitive dysfunction, glaucoma and stroke.

In certain embodiments, patient with hypertension has elevation of both systolic and diastolic blood pressure with the presence of acute target organ disease. These can include pulmonary edema, cardiac ischemia, neurologic deficits, acute renal failure, aortic dissection, eclampsia, and any unknown reason.

In certain embodiments, patient with hypertension has blood pressure values of about 140 mm Hg/90 mm Hg or higher. In certain embodiments, patient with hypertension has blood pressure values higher than 180 mm Hg/120 mm Hg requiring immediate medical attention to prevent organ damage (e.g., in hypertensive crisis). In certain embodiments, patient with hypertension has diastolic blood pressure higher than 120 mm Hg (e.g., in hypertensive urgency).

In certain embodiments, the nasal antihypertensive compositions of the disclosure may be used as replacement for conventional dosage forms containing an antihypertensive agent, e.g., conventional oral tablets or oral solution, for convenience and ease of administration in hypertensive subjects.

In certain embodiments, the antihypertensive drug is selected from the group comprising angiotensin converting enzyme inhibitor (ACEi), angiotensin receptor blocker (ARB), diuretic, β-blocker, calcium channel blocker, central α-agonist, peripheral α-antagonist, direct vasodilator, neprilysin inhibitor, and mixtures thereof.

In certain embodiments, the nasal antihypertensive composition is a nasal solution administered as spray or drops. In certain embodiments, the nasal composition is a nasal suspension or emulsion administered as a spray or drops. The solution, suspension, or emulsion is administered at least once-a-day in one or both nostrils as single spray or drop/nostril, two sprays or drops/nostril, three sprays or drops/nostril, or four sprays or drops/nostril. In certain embodiments, the nasal composition is administered once or twice-a-day as a single or two sprays or drops/nostril.

In certain embodiments, the nasal antihypertensive composition comprising a nasal suspension is administered at least once-a-day in one or both nostrils as pressurized metered aerosol administered once-a-day or twice-a-day in one or both nostrils as single pressurized spray/nostril, two pressurized sprays/nostril, three pressurized sprays/nostril, or four pressurized sprays/nostril.

In certain embodiments, the nasal antihypertensive suspension for administration as pressurized metered aerosol comprises a slurry of the antihypertensive drug.

In certain embodiments, the nasal antihypertensive composition is administered as a dry powder suitable for nasal administration via powder exhalation delivery system (EDS) or a turbuhaler. In certain embodiments, the dry powder compositions are supplied in medication capsules (containing antihypertensive drug) pre-filled in a nosepiece section of the EDS. Such delivery systems can be used to deliver the antihypertensive drug via nose using a reusable device body incorporating a flexible mouth piece to adjust individual anatomic variations, and a piercing assembly to pierce the medication capsule. In certain embodiments, the dry powder composition containing the antihypertensive drug is delivered using a device with a reservoir (e.g., turbuhaler).

### Method of Manufacture

In certain embodiments, the nasal antihypertensive solutions of the disclosure are manufactured by adding required ingredients with mixing in the following order: about 60% of sterile water for injection, weak acid, antihypertensive drug, thickening agent, humectant, salt of the weak acid, and remaining water for injection. The final clear solution is filtered through an aseptic 0.2 μm filter and stored in a sterile holding vessel under nitrogen blanket until filled into sterile bottles/vials and capped with a sterile spray/pump device.

In certain embodiments, the nasal antihypertensive suspensions of the disclosure are manufactured by adding required ingredients with mixing in the following order: about 30% of sterile water for injection, mucoadhesive thickening agent, antihypertensive drug (optionally, micronized or nanosized), osmotic agent, buffer, preservative, humectant, surfactant, and remaining water for injection. This mixture is stirred using a homogenizer to obtain a uniform suspension. The suspension is stored in a holding vessel under nitrogen blanket until filled into sterile bottles/vials and capped with a sterile spray/pump device.

In certain embodiments, the nasal antihypertensive emulsions of the disclosure are manufactured by adding required ingredients with homogenization in the following order: oil, emulsifying agents, antihypertensive drug (optionally, micronized or nanosized) and about 30% sterile water, osmotic agent, buffer, preservative, and remaining sterile water. The emulsion is stored in a holding vessel under nitrogen blanket until filled into sterile bottles/vials and capped with a sterile spray/pump device.

In certain embodiments, the nasal antihypertensive dry powder compositions of the disclosure are manufactured by micronizing or nanosizing the antihypertensive drug, mixing with an inert excipient and filling the micronized or nanonized drug into appropriate capsules for the powder exhalation delivery system, or a reservoir of a turbuhaler device or other powder delivery devices.

In certain embodiments, the nasal antihypertensive pressurized metered compositions of the disclosure are manufactured by micronizing or nanosizing the antihypertensive drug and adding to dehydrated ethanol in a closed vessel with an inbuilt homogenizer to obtain a slurry. The resulting slurry is further homogenized with addition of a propellent to obtain a suspension, which is then filled into canisters with valves and actuators.

### EXAMPLES

### Example 1: Nasal Spray Solution Containing Lisinopril Dihydrate (Preservative-Free Solution)

Lisinopril dihydrate: 65.4 mg (Eq to 60 mg Lisinopril)
HPMC E50 (low viscosity hydroxypropyl methylcellulose): 22 mg
Glycerin (0.5%): 0.005 mL
Citric acid: 1.92 mg
Trisodium citrate: 5.88 mg
Purified water: q.s. to 1 ml.

### Manufacturing Procedure:

1. About 60% of sterile water for injection was added to a clean stainless-steel container.
2. Citric acid was added with continuous stirring to the stainless-steel container from step 1 to obtain a solution.
3. Lisinopril dihydrate was added with continuous stirring to the solution from step 2.
4. HPMC E50 was added with continuous stirring to the solution with lisinopril dihydrate from step 3.
5. Glycerin solution was added with continuous stirring to the solution from step 4.
6. Trisodium citrate was added with continuous stirring to the solution from step 5 and further stirred to obtain a clear solution.
7. Remaining 40% of sterile water for injection was added to the solution from step 6 and stirred to obtain a uniform clear solution.
8. The solution from step 7 was filtered through a sterile 0.2 μm filter into a stainless-steel vessel under nitrogen atmosphere.
9. The sterile solution was filled into sterile glass bottles and capped with sterile spray device—cartridge pump system (CPS).

### Example 2: Nasal Spray Solution Containing Lisinopril Dihydrate

Lisinopril dihydrate: 65.4 mg (Eq to 60 mg Lisinopril)
HPMC E50: 22 mg
Benzalkonium chloride: 1.2 mg
Edetate sodium: 2 mg
Citric acid: 1.92 mg
Trisodium citrate: 5.88 mg
Sodium hydroxide (IN): q.s.
Purified water: q.s. to 1 ml.

### Manufacturing Procedure:

1. About 60% of sterile water for injection is added to a clean stainless-steel container.
2. Citric acid is added with continuous stirring to the stainless-steel container from step 1 to obtain a solution.
3. Lisinopril dihydrate is added with continuous stirring to the solution from step 2.
4. HPMC E50 is added with continuous stirring to the solution with lisinopril dihydrate from step 3.
5 Benzalkonium chloride and Edetate sodium are added with continuous stirring to the solution from step 4 to obtain a uniform solution.
6. Trisodium citrate is added with continuous stirring to the solution from step 5 and further stirred to obtain a clear solution.
7. Sodium hydroxide is added to the clear solution from step 6 to adjust the pH from about 6.1 to about 6.7.
8. Remaining 40% of sterile water for injection is added to the solution from step 7 and stirred to obtain a uniform clear solution.
9. The solution from step 8 is filtered through a sterile 0.2 μm filter into a stainless-steel vessel under nitrogen atmosphere, if required.
10. The sterile solution is filled into glass bottles and capped with spray/pump device.

### Example 3: Nasal Spray Solution Containing Lisinopril Dihydrate (Preservative-Free Solution)

Lisinopril dihydrate: 65.4 mg (Eq to 60 mg lisinopril)
HPMC E50: 22 mg
Sodium phosphate, dibasic: 4.2 mg
Sodium phosphate, monobasic: 4.75 mg
Sodium hydroxide or HCI (IN solution): q.s.
Purified water: q.s. to 1 ml.

### Manufacturing Procedure:

1. About 30% of water was heated to about 80-90° C. in a stainless-steel vessel.
2. HPMC E50 was added with continuous stirring to the heated water from Step 1 to obtain a solution.
3. Another 30% of water was added with stirring to the solution from Step 2 and mixed vigorously.
4. Sodium phosphate, dibasic and sodium phosphate, monobasic were added with stirring.
5. Micronized or nanonized Lisinopril dihydrate, was added with stirring to the solution from step 4, and the mixture was further stirred until a uniform solution was obtained.
6. Sodium hydroxide or hydrochloric acid solution was added, if necessary. to adjust the pH of the solution in the range of pH 6.1 to 6.7
7. Remaining water was added while stirring to the solution from Step 6 to q.s. to 1 ml and stirred to obtain a clear solution
8. The solution from step 7 was filtered through a sterile 0.2 μm filter into a stainless-steel vessel, and stored under nitrogen atmosphere, if necessary.
9. The sterile solution is filled into glass bottles and capped with spray/pump device (CPS).

### Example 4: Stability of Nasal Lisinopril Dihydrate Solution under Controlled Room Temperature and Accelerated Storage Conditions

Following Table 1 provides storage stability of nasal lisinopril solution from Example 1.

TABLE 1

| | | | 40° C./75% RH | | | 25° C./60% RH |
|---|---|---|---|---|---|---|
| Test | Specification | Initial | 1 Month | 2 Month | 3 Month | 3 Month |
| Description | Clear, colorless, | Complies | Complies | Complies | Complies | Complies |
| | slightly viscous | | | | | |
| | solution | | | | | |
| Assay | 90-110% | 99.5% | 98.6 | 101.2 | 99.8 | 99.1% |
| pH | 5-6 | 5.14 | 5.23 | 5.19 | 5.27 | 5.26 |
| Osmolality (mOsmol/Kg) | 270-330 | 301 | 281 | 280 | 279 | 282 |
| Viscosity (cP) | 50-75 | 64.47 | 66.05 | 68.05 | 58.21 | 65.35 |
| N-alkyl-L-lysine | NMT 0.3% | 0.01 | 0.03 | 0.09 | 0.13 | 0.02 |
| DL-homophenylalanine | NMT 0.3% | 0.05 | 0.03 | 0.03 | 0.04 | 0.03 |
| Lisinopril epimer | NMT 0.3% | 0.01 | ND | ND | ND | ND |
| Lisinopril cyclohexyl | NMT 0.3% | ND | ND | ND | ND | ND |
| analog | | | | | | |
| S,S,S-diketopiperazine | NMT 0.3% | ND | 0.09 | 0.18 | 0.28 | 0.04 |
| (Lisinopril related | | | | | | |
| compound A) | | | | | | |
| R,S,S-diketopiperazine | NMT 0.3% | ND | ND | ND | ND | ND |
| N-alkyl lisinopril | NMT 0.15% | ND | ND | ND | ND | ND |
| Individual unspecified | NMT 0.1% | 0.04 | 0.05 | 0.03 | 0.28 | 0.06 |
| impurities | | | | | | |
| Total impurities | NMT 1% | 0.11 | 0.23 | 0.39 | 0.88 | 0.19 |

The data from Table 1 shows that the nasal lisinopril solution at pH of ˜5.2 exhibited an assay of 90-110% of the target concentration and showed acceptable stability with respect to the diketopiperazine (DKP) impurity under controlled room temperature (25° C./60% RH) and accelerated storage condition (40° C./75% RH). Further, no microbial growth was observed initially and during the course of stability testing.

### Example 5: Nasal Spray Suspension Containing Ramipril

Ramipril: 1 mg-20 mg
Sodium Chloride: 0 mg-9 mg
Sodium dihydrogen phosphate: 0 mg-50 mg
Benzalkonium chloride: 0.1 mg-3 mg
HPMC: 0 mg-20 mg
Glycerin: 0-30 mg
Polysorbate 80:0 mg-0.5 mg
Propylene glycol: 0 ml-0.5 ml
Purified water: q.s. to 1 ml.

### Manufacturing Procedure:

1. About 30% of water is heated to about 80-90° C.
2. HPMC is added to the heated water from Step 1 and mixed to obtain a solution.
3. 30% of water is added to the mixture from Step 2 and homogenized.
4. Micronized or nanonized Ramipril, followed by sodium chloride, sodium dihydrogen phosphate, benzalkonium chloride, glycerol, polysorbate 80, and propylene glycol, are added with stirring, to the solution from step 3, and the mixture is further homogenized until a uniform suspension is obtained.
5. Remaining water is added while mixing to the suspension from Step 4 to q.s. to 1 ml.
6. The above suspension is filled into glass bottles and capped with spray device.

### Example 6: Nasal Spray Suspension Containing Olmesartan Medoximil and/or Candesartan Cilexetil

Olmesartan Medoximil: 10 mg-40 mg, Or
Candesartan Cilexetil: 0.1 mg-32 mg
Sodium Chloride: 0 mg-9 mg
Sodium dihydrogen phosphate: 0 mg-50 mg
Benzalkonium chloride: 0.1 mg-3 mg
HPMC: 0 mg-20 mg
Glycerin: 0-30 mg
Polysorbate 80:0 mg-0.5 mg
Propylene glycol: 0 ml-0.5 ml
Purified water: q.s. to 1 ml.

### Manufacturing Procedure:

1. About 30% of water is heated to about 80-90° C.
2. HPMC is added to the heated water from Step 1 and mixed to obtain a solution.
3. 30% of water is added to the mixture from Step 2 and homogenized.
4. Micronized or nanonized Olmesartan medoximil or Candesartan cilexetil, followed by sodium chloride, sodium dihydrogen phosphate, benzalkonium chloride, glycerol, polysorbate 80, and propylene glycol, are added with stirring, to the solution from step 3, and the mixture is further homogenized until a uniform suspension is obtained.
5. Remaining water is added while mixing to the suspension from Step 4 to q.s. to 1 ml.
6. The above suspension is filled into glass bottles and capped with spray device.

### Example 7: Nasal Spray Emulsion Containing Olmesartan Medoximil and/or Candesartan Cilexetil

Olmesartan Medoximil: 10 mg-40 mg OR
Candesartan Cilexetil: 0.1 mg-32 mg
Medium-chain triglycerides (MCT): 0.1 mg-40 mg
Lecithin: 0.1 mg-20 mg
Sodium Chloride: 0 mg-9 mg
Sodium dihydrogen phosphate: 0 mg-50 mg
Propylparaben: 0.1 mg-0.2 mg
Benzalkonium chloride: 0.01 mg-0.3 mg
Purified water: q.s. to 1 ml.

### Manufacturing Procedure:

1. Lecithin is added to MCT with mixing.
2. Micronized or nanonized Olmesartan medoxomil or candesartan cilexetil and 30% of water is added to the mixture from step 1, followed by addition of sodium chloride, sodium dihydrogen phosphate, propylparaben and benzalkonium chloride, and homogenized until a fine emulsion is obtained.
3. Remaining water is added while mixing to the emulsion from Step 2 to q.s. to 1 ml.
4. The above emulsion is filled into glass bottles and capped with spray device.

### Example 8: Nasal Spray Suspension Containing Telmisartan Sodium

Telmisartan Sodium: 0.1 mg-80 mg
Sodium Chloride: 0 mg-9 mg
Sodium dihydrogen phosphate: 0 mg-50 mg
Methylparaben: 0.25 mg-0.7 mg
Propylparaben: 0.1 mg-0.2 mg
Hydroxypropyl methylcellulose: 0 mg-20 mg
Glycerol: 0 mg-30 mg
Purified water: q.s. to 1 ml.

### Manufacturing Procedure:

1. About 30% of water is heated to about 80-90° C.
2. HPMC is added to the heated water from Step 1 and mixed to obtain a solution.
3. Another 30% of water is added to the solution from Step 2 and mixed vigorously.
4. Micronized or nanonized Telmisartan Sodium, followed by sodium dihydrogen phosphate, methylparaben, propylparaben, and glycerol, are added with stirring to the solution from step 3, and the mixture is further homogenized until a uniform suspension is obtained using a homogenizer.
5. Remaining water is added while stirring to the suspension from Step 4 to q.s. to 1 ml.
6. The above suspension is filled into glass bottles and capped with spray device.

### Example 9: Nasal Spray Solution Containing Lisinopril Dihydrate

Lisinopril dihydrate: 1 mg-40 mg
Sodium chloride: 0 mg-9 mg
Sodium dihydrogen phosphate: 0 mg-50 mg
Benzalkonium chloride: 0.1 mg-3 mg
Edetate sodium: 0.01 mg-0.2 mg
Hydroxypropyl methylcellulose: 0 mg-20 mg
Glycerol: 0 mg-30 mg
Purified water: q.s. to 1 ml.

### Manufacturing Procedure:

1. About 30% of water is heated to about 80-90° C.
2. HPMC is added to the heated water from Step 1 and mixed to obtain a solution.
3. Another 30% of water is added to the solution from Step 2 and mixed vigorously.
4. Lisinopril, followed by sodium chloride, benzalkonium chloride, edetate sodium and glycerol, are added with stirring, to the solution from step 3, and the mixture is further stirred until a uniform solution is obtained.
5. Remaining water is added while stirring to the solution from Step 4 to qs to 1 ml.
6. This solution is stored under a nitrogen blanket, if required, until filled into bottles and capped with the pump/spray device.

### Example 10: Nasal Aerosol Composition Containing Lisinopril Dihydrate or Ramipril

Lisinopril Dihydrate: 40 mg-80 mg or
Ramipril: 0.1 mg-20 mg
Alcohol, dehydrated: q.s.
Propellant: Hydroxyfluoroalkanes (HFA 134a or 227 or 152a), Hydrofluoroolefin (HFO-1234ze), butane, nitrogen, carbon dioxide or air.

### Manufacturing Procedure:

1. Micronized or nanonized, free flowing ramipril or lisinopril dihydrate is added to dehydrated alcohol in a suitable closed vessel with inbuilt homogenizer to obtain a homogenized slurry.
2. Propellant is added to the homogenized slurry from step 1 to obtain a suspension.
3. The suspension from step 2 is filled into canisters crimped with valves and actuators.

### Example 11: Nasal Powder Composition Containing Lisinopril Dihydrate or Ramipril

Lisinopril Dihydrate: 40 mg-80 mg or
Ramipril: 0.1 mg-20 mg
HPMC E15: 0 mg-20 mg.

### Manufacturing Procedure:

1. Micronized or nanonized, free flowing ramipril or lisinopril dihydrate is mixed intimately with HPMC using a high shear mixer to obtain a uniform blend.
2. The blend from step 1 is filled into gelatin or hydroxypropyl methyl cellulose or starch or pullalan (naturally fermented tapioca) capsules for use with exhalation delivery system (EDS) device or similar device or in a reservoir for a turbuhaler device.

### Example 12: Pharmacokinetics of Nasal Spray Lisinopril Solution

A single dose pharmacokinetic (PK) study comprising one spray/nostril was conducted in healthy volunteers under fasting conditions to evaluate PK performance of nasal spray solution of antihypertensive drug, e.g., Lisinopril dihydrate solution from Example 1. An open label, balanced, single dose study was conducted in 12 normal, healthy, adult human subjects under fasted conditions. Each patient, following an overnight fast for at least 10 hours, was administered one spray/nostril comprising 6 mg lisinopril equivalent/100 μl/nostril (total two sprays). Blood samples were drawn at various time points until 24 hours post dose.

FIG. 1/Table 2 compare systemic bioavailability of 12 mg lisinopril equivalent dose of aqueous test composition, administered as nasal spray comprising 6 mg lisinopril equivalent/100 μL/nostril in each of the two nostrils, to an equivalent oral dose of marketed lisinopril product (literature data for 10 mL marketed lisinopril oral solution (1 mg/mL)), when administered under fasting conditions.

Data from FIG. 1/Table 2 clearly demonstrates that aqueous lisinopril nasal spray composition provides faster systemic absorption compared to marketed lisinopril oral solution. The aqueous lisinopril composition administered as nasal spray provides a post-dose lag time to systemic drug absorption that is about 1.5 hours less than the lag time to the systemic drug absorption from marketed lisinopril oral solution. Data from Table 2 shows about 9-fold increase in lisinopril plasma concentration from the aqueous lisinopril nasal spray composition of the disclosure compared to lisinopril plasma concentration from marketed oral lisinopril solution at about 1.5 hours post-dose.

TABLE 2

| Time (hr) | Nasal Spray (ng/mL) | Oral Solution (ng/mL) |
|---|---|---|
| 0.00 | 0.00 | — |
| 0.08 | 1.95 | — |
| 0.17 | 3.78 | — |
| 0.25 | 6.36 | — |
| 0.33 | 8.60 | — |
| 0.42 | 11.11 | — |
| 0.50 | 12.03 | — |
| 0.75 | 15.16 | — |
| 1.00 | 18.02 | — |
| 1.25 | 20.28 | — |
| 1.5 | 21.15 | 1.82 |
| 1.75 | 22.10 | |
| 2.00 | 23.30 | |
| 2.50 | 25.59 | 25.61 |
| 3.00 | 25.86 | — |
| 4.00 | 29.88 | 34.15 |
| 6.00 | 33.63 | 42.51 |
| 8.00 | 33.06 | 39.43 |
| 10.00 | 29.48 | 33.55 |
| 12.00 | 25.80 | 25.94 |
| 24.00 | 9.09 | 13.82 |
| 24.20 | — | 7.42 |
| 36.16 | — | 2.69 |
| 48.09 | — | 1.59 |
| 72.04 | — | 1.29 |
| 83.97 | — | 0.92 |
| 96.20 | — | 1.09 |

Table 3 provides PK parameters for the 12 subjects dosed with lisinopril dihydrate nasal solution from Example 1.

TABLE 3

| | Cmax | Tmax | AUC (0-24) | AUC (0-∞) | T½ |
|---|---|---|---|---|---|
| Subject | (ng/mL) | (hrs) | (nghr/mL) | (nghr/mL) | (hrs) |
| 1 | 48.2 | 8.0 | 743 | 990 | 10.07 |
| 2 | 21.1 | 6.0 | 321 | 376 | 7.65 |
| 3 | 36.5 | 0.42 | 195 | 221 | 10.16 |
| 4 | 84.2 | 2.5 | 825 | 905 | 6.70 |
| 5 | 29.7 | 6.0 | 439 | 515 | 7.64 |
| 6 | 23.4 | 8.0 | 345 | 422 | 8.56 |
| 7 | 21.7 | 1.25 | 333 | 381 | 7.21 |
| 8 | 37.8 | 8.0 | 577 | 705 | 8.15 |
| 9 | 29.4 | 8.0 | 394 | 474 | 8.03 |
| 10 | 29.9 | 6.0 | 467 | 588 | 9.78 |
| 11 | 56.8 | 8.0 | 814 | 961 | 7.32 |
| 12 | 65.5 | 8.0 | 1052 | 1282 | 8.37 |
| Mean | 40.01 | 5.85 | 542.11 | 651.68 | 8.30 |
| SD | 19.91 | 2.86 | 260.41 | 318.61 | 1.15 |
| % CV | 50 | 49 | 48 | 49 | 14 |
| Median | 33.18 | 7.00 | 453.00 | 551.69 | 8.09 |
| Min | 21.09 | 0.42 | 194.63 | 220.0 | 6.7 |
| Max | 84.18 | 8.00 | 1051.76 | 1282.22 | 10.16 |
| N | 12 | 12 | 12 | 12 | 12 |

### Example 13: Nasal Spray Suspension Containing Amlodipine

Amlodipine besylate: 2.5 mg-10 mg
Sodium Chloride: 0 mg-9 mg
Sodium dihydrogen phosphate: 0 mg-50 mg
Benzalkonium chloride: 0.1 mg-3 mg
HPMC: 0 mg-20 mg
Glycerin: 0-30 mg
Polysorbate 80:0 mg-0.5 mg
Propylene glycol: 0 ml-0.5 ml
Purified water: q.s. to 1 ml.

### Manufacturing Procedure:

1. About 30% of water is heated to about 80-90° C.
2. HPMC is added to the heated water from Step 1 and mixed to obtain a solution.
3. 30% of water is added to the solution from Step 2 and homogenized to obtain a homogenized suspension.
4. Micronized or nanonized amlodipine besylate, followed by sodium chloride, sodium dihydrogen phosphate, benzalkonium chloride, glycerol, polysorbate 80, and propylene glycol, are added with stirring, to the homogenized suspension from step 3, and the suspension is further homogenized until a uniform suspension is obtained.
5. Remaining water is added while mixing to the suspension from Step 4 to q.s. to 1 ml.
6. The suspension from step 5 is filled into glass bottles and capped with a spray device.

### Example 14: Nasal Spray Solution Containing Clonidine

Clonidine: 0.05 mg-0.6 mg
HPMC E50 (low viscosity hydroxypropyl methylcellulose): 22 mg
Glycerin (0.5%): 0.005 mL
Citric acid: 1.92 mg
Sodium Chloride: 2 mg
Sodium hydroxide (IN solution): q.s.
Purified water: q.s. to 1 ml.

### Manufacturing Procedure:

1. About 30% of water is heated to about 80-90° C. in a stainless-steel vessel.
2. HPMC E50 is added with continuous stirring to the heated water from Step 1 to obtain a solution.
3. Another 30% of water is added with stirring to the solution from Step 2 and mixed vigorously.
4. Micronized or nanonized Clonidine hydrochloride, followed by citric acid, sodium chloride, and glycerin, are added with stirring, to the solution from step 3, and the mixture is further stirred until a uniform solution is obtained.
5. Sodium hydroxide solution is added, if required, to adjust the pH of the solution in the range of pH 6.1 to 6.7
6. Remaining water is added while stirring to the solution from Step 5 to q.s. to 1 ml and stirred to obtain a clear solution
7. The solution from step 6 is filtered through a sterile 0.2 μm filter into a stainless-steel vessel under nitrogen atmosphere, if necessary.
8. The sterile solution from step 7 is filled into glass bottles and capped with a spray/pump device (CPS).

1-20. (canceled)

21. An aqueous solution composition for nasal administration comprising lisinopril or a hydrate thereof, and a buffer,
wherein the composition is free of a penetration enhancer,
wherein the composition on nasal administration to a person in need thereof provides lisinopril pharmacokinetic profile comprising AUCO-∞ (area under the curve from 0-∞ time points) of about 651.68±318.61 (49) ng hr/mL, wherein the pharmacokinetic profile is expressed as mean value±standard deviation (% variance),
wherein the composition is administered in both nostrils as a single spray/nostril comprising 6 mg lisinopril equivalent/100 μL solution, and providing a total administered dose of 12 mg lisinopril equivalent.

22. The solution composition of claim 21, wherein the composition provides a maximum lisinopril plasma concentration (Cmax) of about 40±20 (50) ng/mL, wherein the plasma concentration is expressed as mean value±standard deviation (% variance).

23. The solution composition of claim 21, comprising lisinopril dihydrate.

24. The solution composition of claim 21, wherein the solution on nasal administration provides a lag time to systemic absorption of lisinopril of 1.5 hours or less.

25. The solution composition of claim 24, wherein the solution on nasal administration provides a lag time to the systemic absorption of lisinopril of 1 hour or less.

26. The solution composition of claim 25, wherein the solution on nasal administration provides a lag time to systemic absorption of lisinopril of about 5 minutes.

27. (canceled)

28. (canceled)

29. The solution composition of claim 21, wherein the solution exhibits a pH of from about 6 to about 7.

30. (canceled)

31. The solution composition of claim 21, wherein the buffer is selected from the group consisting of sodium dihydrogen phosphate; disodium hydrogen phosphate; sodium citrate and citric acid; sodium acetate, acetic acid; maleic acid and salts; carbonic acid, ammonia, carbonate, sodium carbonate, sodium bicarbonate, trisodium citrate, lactic acid, maleic acid, tartaric acid, sodium tartrate and salts, glycine, tris (hydroxymethyl) aminomethane, and mixtures thereof.

32. The solution composition of claim 21, further comprising a mucoadhesive agent selected from the group consisting of carboxymethylcellulose, sodium carboxymethylcellulose, methyl cellulose, hydroxypropyl methylcellulose, hydroxypropyl cellulose, hydroxyethyl cellulose, microcrystalline cellulose, mixture of microcrystalline cellulose and carboxymethylcellulose sodium, poloxamer, polyvinyl alcohol, polyvinyl pyrrolidone, polyvinyl acetate, polydextrose, pectin, chitosan, acacia, polyethylene glycol, sodium alginate, bentonite, carbomer, carrageenan and, guar gum, alginic acid, carbomer, hectorite, povidone, tragacanth, xanthan gum and mixtures thereof.

33. The solution composition of claim 21, wherein the composition is a preservative free composition.

34. The solution composition of claim 21, further comprising a tonicity adjusting agent is selected from the group consisting of sodium chloride, dextrose, glycerin, potassium chloride, mannitol, calcium chloride, glucose, glycine, magnesium chloride, potassium chloride, sorbitol, sucrose, sodium sulfate, and mixtures thereof.

35. The solution composition of claim 21, further comprising a pH adjusting agent is selected from the group consisting of hydrochloric acid, sulfuric acid, and sodium hydroxide.

36. The solution composition of claim 21, further comprising a humectant selected from the group consisting of glycerol, propylene glycol, sorbitol, triacetin, mannitol, sucrose, glucose, allantoin, and mixtures thereof.)

37. (canceled)

38. The solution composition of claim 23 comprising from about 4 mg/100 μl to about 8 mg/100 μl lisinopril dihydrate.

39. An aqueous solution composition for nasal administration comprising 4 mg/100 μl to about 8 mg/100 μl lisinopril dihydrate, a buffer, a mucoadhesive thickening agent, and a humectant, wherein the composition is a preservative free,
wherein the composition is stored in a spray/pump device, and
wherein the composition comprises at least 90% wt/vol of lisinopril dihydrate on storage for at least about 3 months at 25° C./60% RH.

40. An aqueous solution composition for nasal administration comprising 4 mg/100 μl to about 8 mg/100 μl lisinopril dihydrate, a buffer, a mucoadhesive agent, and a humectant, wherein
the composition does not include a penetration enhancer,
wherein the composition on nasal administration provides a lag time to systemic absorption of lisinopril of less than about 1.5 hours.

41. The composition of claim 40, wherein the composition is stored in a spray/pump device.

42. The composition of claim 39, wherein the composition does not show any microbial growth on storage for at least about 3 months under controlled room temperature (25° C./60% RH) condition.
